(19) United States
(12) Patent Application Publication
(10) Pub. No.: US 2020/0390398 A1
(43) Pub. Date: Dec. 17, 2020

(54) Toilet with User Detection

(71) Applicant: Medic, Inc., Provo, UT (US)

(72) Inventor: David R. Hall, Provo, UT (US)

(73) Assignee: Medic, Inc., Provo, UT (US)

(21) Appl. No.: 16/903,945

(22) Filed: Jun. 17, 2020

Related U.S. Application Data
(63) Continuation-in-part of application No. 16/578,118, filed on Sep. 20, 2019.
Application No. 16/578,118 is a continuation-in-part of application No. 16/709,163, filed on Dec. 10, 2019.
(60) Provisional application No. 62/862,278, filed on Jun. 17, 2019.
(60) Provisional application No. 62/862,284, filed on Jun. 17, 2019.
(60) Provisional application No. 62/862,603, filed on Jun. 17, 2019.
(60) Provisional application No. 62/862,564, filed on Jun. 17, 2019.
(60) Provisional application No. 62/888,683, filed on Aug. 19, 2019.
(60) Provisional application No. 62/907,408, filed on Oct. 9, 2019.
(60) Provisional application No. 62/907,432, filed on Sep. 27, 2019.
(60) Provisional application No. 62/862,662, filed on Jun. 17, 2019.
(60) Provisional application No. 62/867,657, filed on Jun. 27, 2019.
(60) Provisional application No. 62/888,968, filed on Aug. 19, 2019.

(51) Int. Cl. A61B 5/00; E03D 9/00; G01G 19/50; A61B 5/0205

(52) U.S. Cl. CPC A61B 5/6891; E03D 9/00; G01G 19/50; A61B 5/04284; A61B 5/0075; A61B 5/0077; A61B 5/02055

(57) ABSTRACT

An analytical toilet comprising a bowl for receiving excreta; a health and wellness sensor; and a processor receiving and analyzing data from the health and wellness sensor; wherein the processor analyzes data from the health and wellness sensor to detect when a user is positioned to use the toilet is disclosed. In one embodiment, a processor analyzes data from the health and wellness sensor is used by the processor to identity a user. In another embodiment, a sensor detector capable of identifying the user.

### CROSS-REFERENCE TO RELATED APPLICATIONS

This application is a continuation in part of U.S. patent application Ser. No. 16/578,118 titled “Device for Providing Health and Wellness Data Through Foot Imaging” filed on 20 Sep. 2019, which claims priority to U.S. Provisional Patent App. Nos. 62/862,278 titled “Device for Assessing Health and Wellness through Foot Imaging” filed on 17 Jun. 2019; 62/862,284 titled “Device for Health and Wellness Assessment by Thermal Imaging of Feet” filed on 17 Jun. 2019; and U.S. 62/862,603 titled “Toilet with Sensors for Imaging Legs and Feet and Assessing Health Conditions Thereby” filed on 17 Jun. 2019, all of which are all incorporated herein by reference in their entireties. This application is a continuation-in-part of U.S. patent application Ser. No. 16/709,163 titled “Toilet with Internal Lifting System” filed on 10 Dec. 2019, which claims priority to U.S. Provisional Patent App. Nos. 62/862,564 titled “Toilet with Frame, Bowl and Shroud” filed on 17 Jun. 2019; 62/888,683 titled “Toilet with Multiple Point Lifting System” filed 19 Aug. 2019; 62/907,408 titled “Toilet with Personalized Lifting System for Bowl and Seat” filed on 9 Oct. 2019; and 62/907,432 titled “Toilet with User Weight Driven Seat Lift Profile” filed on 27 Sep. 2019, all of which is incorporated herein by reference in their entireties. This application claims priority to U.S. Provisional Patent Application Nos. 62/862,662 titled “User Detection Using Smart Toilet Sensors” filed on 17 Jun. 2019; 62/888,968 titled “Toilet with Foot-Based User Recognition” filed on 19 Aug. 2019; and 62/867,657 titled “Method for Verification of Volume Data in Urinalysis Toilet” filed 17 Jun. 2019, each of which is incorporated herein by reference in its entirety.

### TECHNICAL FIELD

The present disclosure relates to analytical toilets. More particularly, it relates to diagnostic medical toilets equipped to provide health and wellness information to the user.

### BACKGROUND

The ability to track an individual's health and wellness is currently limited due to the lack of available data related to personal health. Many diagnostic tools are based on examination and testing of excreta, but the high cost of frequent doctor's visits and/or scans make these options available only on a very limited and infrequent basis. Thus, they are not widely available to people interested in tracking their own personal wellbeing.

Toilets present a fertile environment for locating a variety of useful sensors to detect, analyze, and track trends for multiple health conditions. Locating sensors in such a location allows for passive observation and tracking on a regular basis of daily visits without the necessity of visiting a medical clinic for collection of samples and data. Monitoring trends over time of health conditions supports continual wellness monitoring and maintenance rather than waiting for symptoms to appear and become severe enough to motivate a person to seek care. At that point, preventative care may be eliminated as an option leaving only more intrusive and potentially less effective curative treatments. An ounce of prevention is worth a pound of cure.

In order to get the most useful data out of a toilet-based system, it is necessary to know the identity of each user to the greatest extent possible. This can be done manually by a user, but users are often hesitant to touch surfaces in a bathroom. This is especially true for surfaces that are touched by other users. Thus, it would be ideal for the system to be able to identify a user without requiring the user to self-identify. However, there are privacy concerns using known technology, such as facial recognition, that utilize cameras in the bathroom.

Just a few examples of smart toilets and other bathroom devices can be seen in the following U.S. Patents and Published Applications: U.S. Pat. No. 9,867,513, entitled “Medical Toilet With User Authentication”; U.S. Pat. No. 10,123,784, entitled “In Situ Specimen Collection Receptacle In A Toilet And Being In Communication With A Spectral Analyzer”; U.S. Pat. No. 10,273,674, entitled “Toilet Bowl For Separating Fecal Matter And Urine For Collection And Analysis”; US 2016/0000378, entitled “Human Health Property Monitoring System”; US 2018/0020984, entitled “Method Of Monitoring Health While Using A Toilet”; US 2018/0055488, entitled “Toilet Volatile Organic Compound Analysis System For Urine”; US 2018/0078191, entitled “Medical Toilet For Collecting And Analyzing Multiple Metrics”; US 2018/0140284, entitled “Medical Toilet With User Customized Health Metric Validation System”; US 2018/0165417, entitled “Bathroom Telemedicine Station.” The disclosures of all these patents and applications are incorporated by reference in their entireties.

### SUMMARY

In a first aspect, the disclosure provides an analytical toilet comprising a bowl for receiving excreta; a health and wellness sensor; and a processor receiving and analyzing data from the health and wellness sensor; wherein the processor analyzes data from the health and wellness sensor to detect when a user is positioned to use the toilet.

In a second aspect, the disclosure provides a toilet that includes a processor that analyzes data from the health and wellness sensor to identity a user.

In a third embodiment, the disclosure provides a toilet that includes a detector that is capable of detecting an identifying item of the user.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a side perspective view of an exemplary embodiment of an analytical toilet with a lid and foot platform according to the present disclosure.

FIG. 2 is a side perspective view of the toilet of FIG. 1 with the toilet lid opened, the foot platform raised, and exemplary handles or armrests raised.

FIG. 3 is a side view of the toilet of FIG. 1 with the outer covering removed to show the interior components with the foot platform lowered.

FIG. 4 is a side view of the toilet of FIG. 1 with the outer covering removed to show the interior components with the foot platform raised.

FIG. 4A is an enlarged cross-sectional view of the seat of FIG. 4.

FIG. 5A is a top view of a second exemplary embodiment of a toilet seat with a floating hinge according to the present disclosure.

FIG. 5B is a side view of the toilet seat of FIG. 5A.

FIG. 5C is a cross-sectional view of the hinge of the toilet seat of FIG. 5A taken within Line C.

FIG. 5D is a cross-sectional view of the hinge of the toilet seat of FIG. 5A taken within Line D in FIG. 5B.

FIG. 6A is a first exploded perspective view of the hinge of the toilet seat of FIG. 5A.

FIG. 6B is a second exploded perspective view of the hinge of the toilet seat of FIG. 5A.

FIG. 7 is a perspective view of an exemplary embodiment of an analytical toilet with a foot platform according to the present disclosure.

FIG. 8 is a perspective view of the foot platform of the toilet of FIG. 1 in a raised position;

FIG. 9 is a perspective view of an analytical toilet with sensors in the seat and lid.

FIG. 10 is a side view of the toilet of FIG. 9 with a user seated thereon.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, “toilet” is meant to refer to any device or system for receiving human excreta, including urinals.

As used herein, the term “bowl” refers to the portion of a toilet that is designed to receive excreta.

As used herein, the term “base” or “frame” refers to the portion of the toilet below and around the bowl supporting it.

As used herein, the term “user” refers to any individual who interacts with the toilet and deposits excreta therein.

As used herein, the term “excreta” refers to any substance released from the body of a user including urine, feces, menstrual discharge, saliva, expectorate, and anything contained or excreted therewith.

As used herein, the term “sensor” is meant to refer to any device for detecting and/or measuring a property of a person or of a substance regardless of how that property is detected or measured, including the absence of a target molecule or characteristic. Sensors may use a variety of technologies including, but not limited to, MOS (metal oxide semiconductor), CMOS (complementary metal oxide semiconductor), CCD (charge-coupled device), FET (field-effect transistors), nano-FET, MOSFET (metal oxide semiconductor field-effect transistors), spectrometers, volume measurement devices, weight sensors, temperature gauges, chromatographs, mass spectrometers, IR (infrared) detector, near IR detector, visible light detectors, and electrodes, microphones, load cells, pressure gauges, PPG (photoplethysmogram), thermometers (including IR and thermocouples), rheometers, durometers, pH detectors, scent detectors gas, and analyzers.

As used herein, the term “imaging sensor” is meant to refer to any device for detecting and/or measuring a property of a person or of a substance that relies on electromagnetic radiation of any wavelength (e.g., visible light, infrared light, xray) or sound waves (e.g., ultrasound) to view the surface or interior of a user or substance. The term “imaging sensor” does not require that an image or picture is created or stored even if the sensor is capable of creating an image.

As used herein, the term “data connection” and similar terms are meant to refer to any wired or wireless means of transmitting analog or digital data and a data connection may refer to a connection within a toilet system or with devices outside the toilet.

As used herein, the terms “biomarker” and “biological marker” are meant to refer to a measurable indicator of some biological state or condition, such as a normal biological processes, pathogenic processes, or pharmacologic responses to a therapeutic intervention. Some biomarkers are related to individual states or conditions. Other biomarkers are related to groups or classifications or states or conditions. For example, a biomarker may be symptomatic of a single disease or of a group of similar diseases that create the same biomarker.

As used herein, the term “analyte” is meant to refer to a substance whose chemical constituents are being identified and measured.

As used herein, the prefix “nano” is meant to refer to something in size such that units are often converted to the nano-scale for ease before a value is provided. For example, the dimensions of a molecule may be given in nanometers rather than in meters.

### Exemplary Embodiments

The present disclosure relates to an analytical toilet capable of performing health and wellness testing on multiple users, including analysis of the contents of the user's excreta. More particularly, it relates to a toilet that uses health and wellness sensors to detect the presence of a user ready to use the toilet and identify the user without active identification by the user.

In a preferred embodiment, the system is able to detect the presence of a user ready to use the sensor. Some smart toilets are able to detect a nearby user entering the bathroom. The disclosed system is designed not to identify a person in the same room unless that person is in position to use the toilet standing or sitting. In a preferred embodiment, the system includes weight sensors able to measure weight on the seat and on a foot platform or scale in front of the toilet. In another preferred system, other sensors, such as photoplethysmograms, on the seat or inner surface of the lid are able to detect the presence of a user on the seat. The seat weight is preferably designed not to register the weight of a user sitting on the closed lid. In various exemplary embodiments, once the toilet system detects a user it responds by activating additional sensors as appropriate, such as by determining whether the user is sitting or standing.

In various exemplary embodiments, some of the sensors are continuously collecting data and storing it in a temporary storage buffer. For example, a PPG or spectrometer may have a thirty second buffer of data that is continuously updated (with the oldest data continuously discarded). The system is able to differentiate data from an idle system from one that is in actual use. For example, the chemical signature of water as seen by a spectrometer is different from that of urine. Thus, when the system detect a change in sensor data indicative of the presence of a user or the occurrence of an excretion event, the system saves the buffered data along with new data as the event progresses both for the sensor that detected the event and other sensors in the toilet system.

In a preferred embodiment, the primary mechanism for identifying a user is by scanning their feet and comparing the results to a database of known users. In various other preferred embodiments, the initial identification is cross-checked with data obtained from other sensors (e.g., weight, photoplethysmogram, etc.) to confirm or reject the initial identification based on comparison to historical measurements for the user. Computer vision is then combined with machine learning to predict the identity of the user based on images of the foot, so that the device can correctly associate a user with the correct data. Proof of concept tests have shown that Medic can achieve 97% accuracy in correctly identifying the owner of a foot. Since the cameras are at foot level and pointed downward, there are fewer privacy concerns than a camera pointed at a face. The image processing may take place by streaming the camera images to the cloud and processing them in the cloud, or the image processing could take place on a dedicated chip on the toilet and the toilet could report the user ID without ever storing the images. The second option would further increase privacy because the images are never stored.

In another embodiment, a detector is able to identify the user by detecting the proximity of an identifying item. Such items include personal electronic devices such as cell phones, credit or debit cards, RFID tags, ID cards, or other personal items. These items are preferably detected using wireless technology such as near-field communication or Bluetooth.

Once the user is identified, specific analytical tools or tests may be activated or deactivated based on the user's known preferences or needs.

In a preferred embodiment, one or more imaging sensors are used to image the feet and an artificial intelligence (AI) program trained with a machine learning algorithm is used to identify the user. The AI is also used to examine the feet for possible indicators of health issues. A system for analyzing feet in this manner is disclosed in U.S. patent application Ser. No. 16/578,118 titled “Device for Providing Health and Wellness Data Through Foot Imaging.” Image processing for identification or other purposes may take place in the toilet system or remotely (e.g., a cloud server).

In a preferred embodiment, the imaging sensors are positioned above the feet so that they are “looking” downward away from the user's body. In some embodiments, imaging sensors are located under the user's feet. These sensors are preferably infrared sensors that cannot “see” the user's features or are focused such that the user above the ankles are out of focus. In another embodiment, any images captured are obfuscated. Such a system is disclosed in International Patent App. No. PCT/US2018/13836 titled “Health Monitoring System Including Privacy-Ensuring Obfuscated Camera Images” filed on 16 Jan. 2018, which is incorporated herein by reference in its entirety.

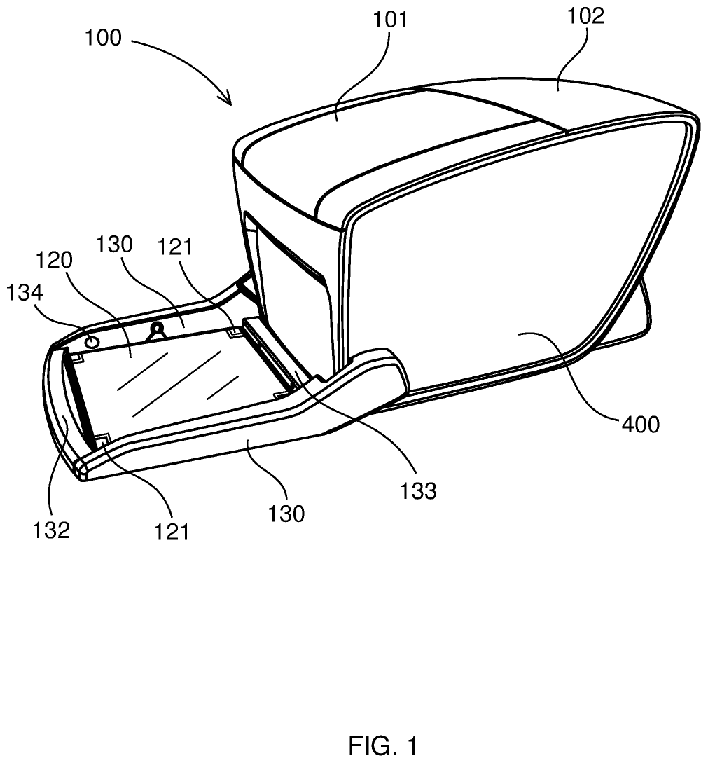

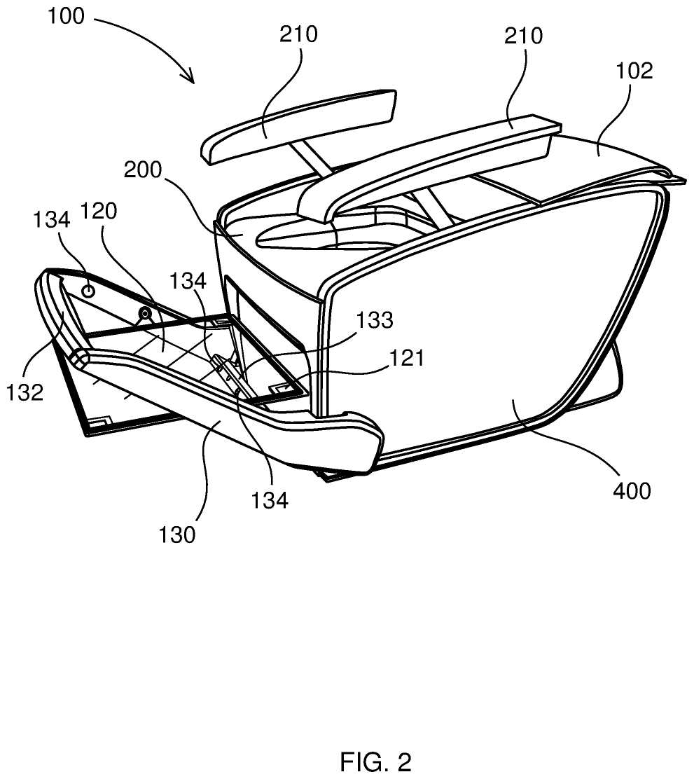

Referring to FIG. 1, an exemplary embodiment of a toilet is shown with a closed lid 101, lowered foot platform 120, and retracted handles 210. FIG. 2 shows the toilet with an open lid 101, raised foot platform 120, and extended handles 210. In a preferred embodiment, handles 210 are adapted to be readily gripped by a user, especially while sitting or rising. In a preferred embodiment, the handles 210 move while the seat 200 is lowering and lifting a user to enable them to grip the handles 210 and use them for support during sitting and rising. The toilet is enclosed with a shroud 400. The foot platform 120 includes weight sensors 121 placed under the foot platform 120 between in and the structure supporting it.

In various exemplary embodiments, the shroud 400 covers all the internal support components that comprise the frame as well as the bowl 300 (i.e., the bowl is covered except for the top that would be covered by a lid). In preferred embodiments, the shroud 400 comprises at least one sections of articulated or flexible (e.g., elastic, material) assembled such that the shroud 400 can contract or expand as the frame 110 moves the bowl 300 and/or seat 200. Other sections of the shroud may be rigid. In various exemplary embodiments, the at least some portions of the shroud are removable or openable to allow access to the interior of the toilet frame. The interior of the toilet frame may include, among other things, plumbing connections, fluid supply lines, support structures, health and wellness assessment devices, electronic circuits, digital devices (e.g., processors, memory), storage tanks, and communication modules. In various preferred embodiments, cover 102 opens to allow access to the interior of the frame.

In various exemplary embodiments, the shrouds 400 are preferably designed for aesthetic and other purposes. For example, the shrouds 400 can be made in various colors to suit the décor of the bathroom the toilet is installed in. Besides colors, the shrouds 400 can include patterns or graphics so that the toilet 100 “makes a statement” in the bathroom.

In various exemplary embodiments, the handles 210 include at least one button, switch, sensor, etc. through which the user may control the seat, including, but not limited to, starting and stopping the seat 200, reversing seat movement direction, adjusting the position or angle of the seat 200, and adjusting the height of the bowl 300, seat 200, or foot platform 120.

In various exemplary embodiments, the handles 210 may include at least one health and wellness sensors. The sensors may be used to measure how hard the user is gripping and provide feedback to the lift control. For example, if a user suddenly increases their grip it is likely that they are struggling with standing or with their balance. Grip pressure may also be tracked over time. The sensors may also include detection for heart rate, body temperature, hand health indicators, light sensors, electrocardiogram, pulse, blood test (e.g., using a finger prick), etc. Toilet handles with health and wellness sensors are disclosed in U.S. Patent Pub. No. 2018/0084959 titled “Instrumented Toilet Seat” filed on 27 Sep. 2016, the entire disclosure of which is incorporated herein by reference in its entirety.

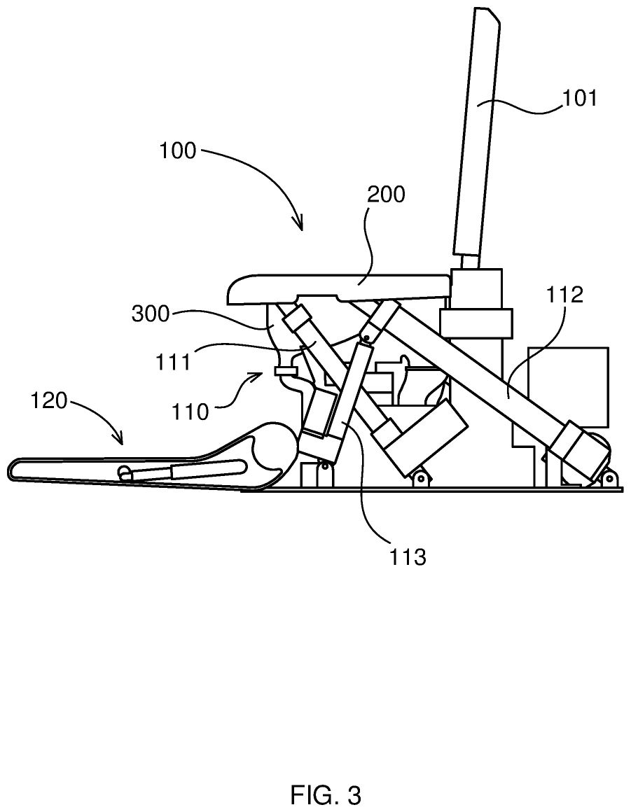

Referring to FIG. 3, an exemplary embodiment of a toilet 100 is shown with a raiseable seat. The toilet 100 includes a frame 110, bowl 300 supported by the frame 110, a lid 101, and a seat 200 also supported by the frame 110. In a preferred embodiment, the seat 200 is supported by two sets of seat actuators 111, 112, and 113. In other embodiments, the number and arrangement of actuators may vary.

In a preferred embodiment, a pair of first seat actuators 111 are pivotally connected to a front area of the seat 200 and a pair of second seat actuators 112 are pivotally connected to a back area of the seat 200 providing support at four “corners” of the seat 200. The four seat actuators 111 and 112 are also pivotally attached to the frame 110. An additional pair of third seat actuators 113 are pivotally attached to the frame 110 at one end and pivotally attached to the second pair of seat actuators 112 at their other end. In another embodiment, the third seat actuators 113 are attached to the first pair of seat actuators 111.

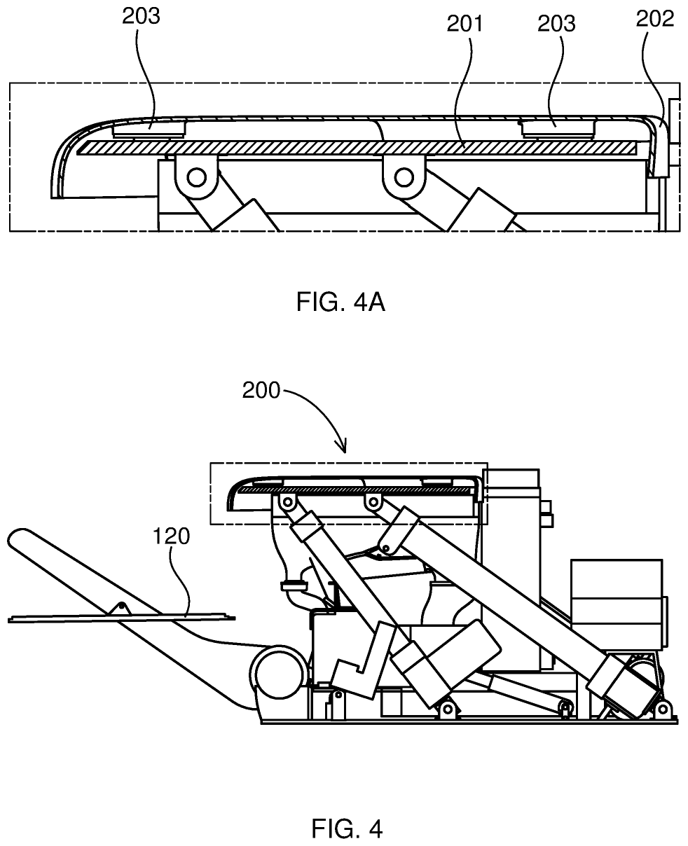

Referring to FIGS. 4 and 4A, an exemplary embodiment of a floating toilet seat 200 according to the present disclosure is shown. The seat includes a rigid seat support 201 covered by a shroud 202. Weight sensors 203 are placed between the seat support 201 and floating cover 202. In various other embodiments, the weight sensors may be placed in a variety of places such as under the actuators or under the frame.

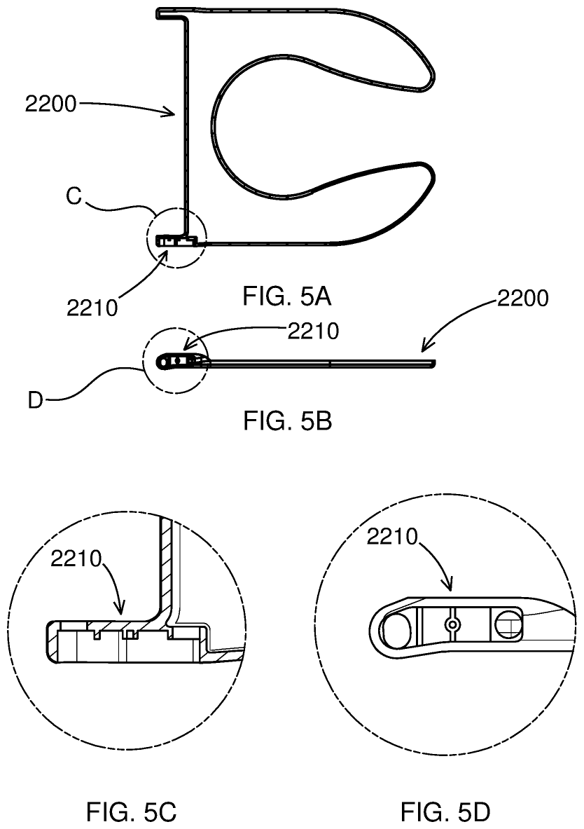

Referring to FIGS. 5A-D, an exemplary embodiment of a toilet seat 2200 with a floating hinge 2210 is shown. FIG. 5D shows the interior of hinge 2210. Some portions of the floating hinge 2210 may be integral to the seat or may be attached to the toilet seat 2200 with connectors (e.g., screws or bolts).

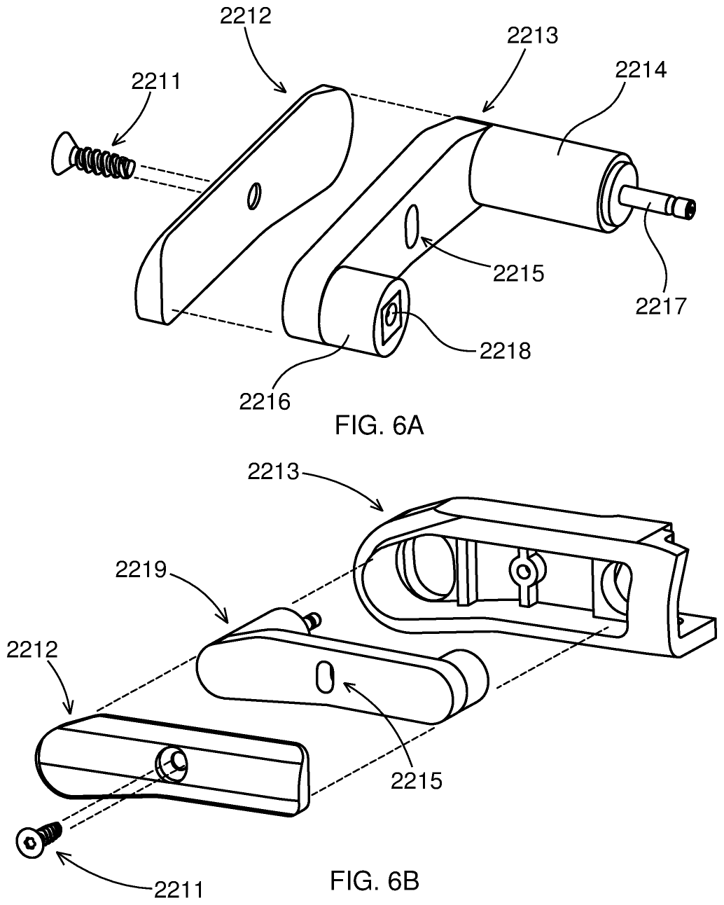

Referring to FIGS. 6A-B, show the components of an exemplary floating hinge 2210. FIG. 6A shows one side of the hinge 2210 assembly. FIG. 6B shows it from the opposite side. In various exemplary embodiments, the hinge 2210 comprises a floating hinge case 2213, pivot bar 2219, and floating hinge cover 2212 connected by screw 2211. Screw 2211 connects the cover 2212 to the case 2213 enclosing the pivot bar 2219. In some embodiments, the screw 2211 also connects the hinge 2210 to the seat 2200.

In various exemplary embodiments, pivoting bar 2219 comprises seat axle 2214, screw slot 2215, pivot axle 2216, and electrical connectors 2217 and 2218. Seat axle 2214 is adapted to connect to the toilet and provides a point around which the seat 2200 is raised and lowered. The pivot axle 2216 rotatably connects the pivot bar 2219 to the hinge case 2213. As will be described in more detail below, the screw slot 2215 allows the pivot bar 2219 to pivot around the pivot axle 2216 without interacting with screw 2211.

In various exemplary embodiments, connectors 2217 and 2218 are included to provide a supply of electrical power to components within the seat, such as load cells, health and wellness sensors, processors, controllers, and wireless communications among others. Connectors 2217 and 2218 may also provide data connections to similar components elsewhere in the system. In preferred embodiments, the electrical wiring connecting connectors 2217 and 2218 are molded into the pivot bar 2219.

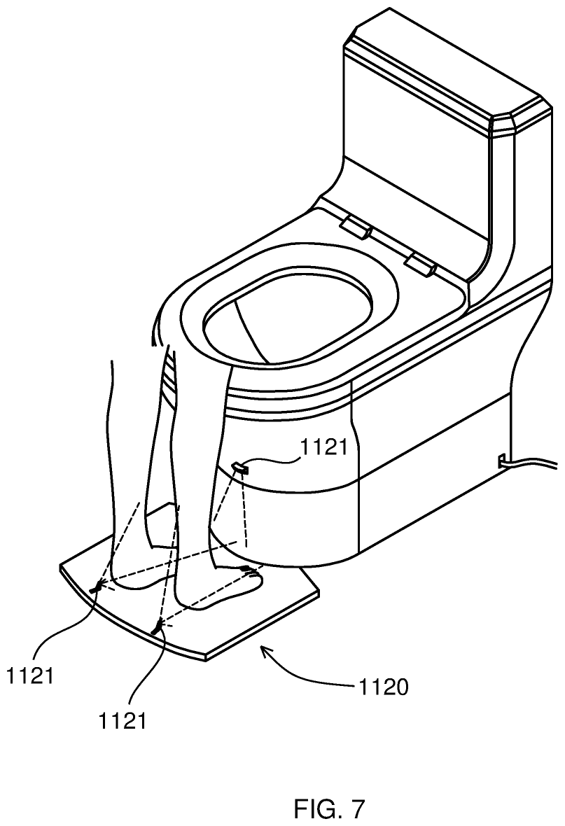

Referring to FIG. 7, a smart toilet including a platform 120 for placement of a user's feet is shown. In various embodiments, imaging sensors may be located at one or more positions around the user's feet. For example, imaging sensors may be placed under the user's feet; to the sides of the user's feet; above and to the front, back or sides of the user's feet; or behind the user's feet. In various other embodiments, the platform 120 may be part of another device, such as a bathroom scale, or may be an area of floor (e.g., in front of a toilet) where one or more sensors are focused. In various exemplary embodiments, the platform 120 may be a part of the floor itself, or a separate unit built into or placed on top of the floor and may include imaging sensors under the portion of the floor comprising the platform 120. The platform 120 is preferably comprised of a transparent material to facilitate the use of imaging sensors under the platform 120 for analyzing the bottom of the user's feet.

In FIG. 7, the user's feet are shown facing the toilet. In various exemplary embodiments, the system is capable of scanning feet regardless of how they are placed on the platform. In various exemplary embodiments, the system is capable of analyzing the image data to determine the orientation of the user's feet and adapt accordingly. However, in most embodiments the imaging sensor arrays are optimized for feet in one or two general positions, typically facing toward or away from a smart toilet.

In the embodiment of FIG. 7, the two imaging sensor arrays 1121 are located behind the feet of a standing user or the front of the feet of a sitting user. In various other exemplary embodiments, sensor arrays 1120 may be placed on or more other sides or in the interior of the platform 1120. In addition to imaging, the sensor arrays 1121 may be used to indicate the presence of a user to the toilet system.

In various exemplary embodiments, the health and wellness sensors are integral to the toilet. In other embodiments, some health and wellness sensors may be in peripheral devices electronically connected to the toilet. In some embodiments, a foot scale may be used in place of a foot platform integral to the toilet. In some embodiments, sensors may be placed around the toilet for analyzing the user (e.g., infrared sensors).

In various exemplary embodiments, one or more of the sensors, or reflecting mirrors, are designed to be in one of two discreet states: one in which they are stored within the profile of the foot platform and a second in which they are in a deployed state where they are in an appropriate position and angle for full viewing and sensing of the foot, ankle, and/or lower leg.

In various exemplary embodiments, the one or more imaging sensors include one or more longwave infrared (LWIR) imaging cameras or thermal cameras. These cameras detect heat differences across the feet that can be indicative of a wide variety of health and wellness issues.

In various exemplary embodiments, the one or more imaging sensors include one or more near infrared (NIR) sensitive cameras and a source of NIR light. By illuminating the veins with NIR light, veins located just below the skin can be detected, evaluated, and tracked. Data gathered over time about veins allows one to track trends and detect changes in size and other characteristics, including varicose veins. This information may be used to monitor indicators for potential blood clots, blood flow, and general vein health.

In various embodiments, the one or more imaging sensors include one or more visual light cameras. Computer vision may be used to process and analyze the images for indicators of many different health and wellness issues. Current images may also be compared to past images to identify changes and indicators of health and wellness issues.

In various exemplary embodiments, the one or more imaging sensors include one or more 3D cameras. 3D cameras are used to gather spatial information and data about the size and shape of the feet. This data can be used to track changes in the size and dimension of the feet indicative of health and wellness problems such as, but not limited to, swelling, water retention, blood circulation deficiencies, bunions, growths, hammer toes and other toe deformities, and arch problems.

In various exemplary embodiments, the one or more imaging sensors include one or more ultrasonic 3D imaging devices. Ultrasound technology may be used to create images of the inside of the foot.

In various exemplary embodiments, the one or more imaging sensors include one or more infrared (IR) cameras. Temperature data can be used to identify hot spots indicative of injury or developing conditions before they manifest noticeable symptoms. It can also be used to monitor general blood flow through the feet and other health and wellness indicators.

In various exemplary embodiments, the one or more sensors may comprise an array of sensors including one or more, including all possible combinations, of the sensors described above.

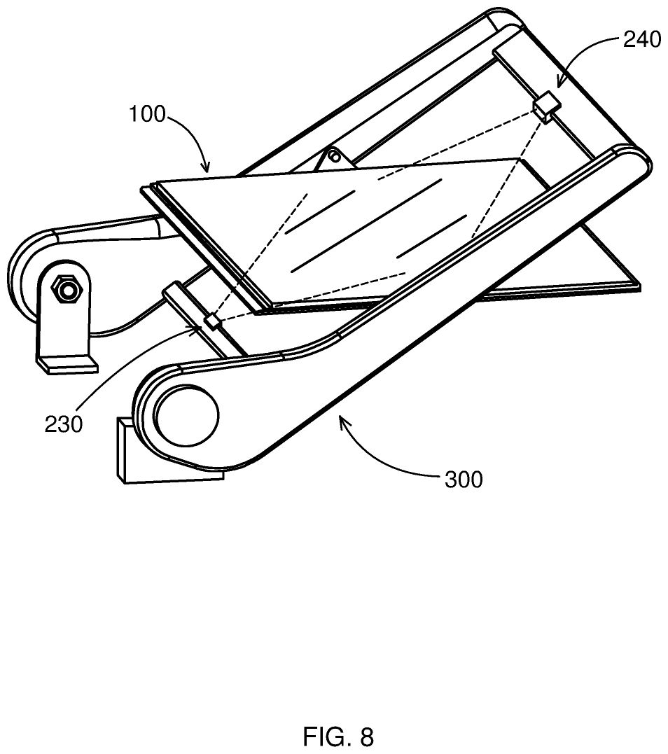

Now referring to FIG. 8, an exemplary embodiment of a toilet foot platform 120 according to the present disclosure is shown. The foot platform 120 includes weight sensors 121 placed under the foot platform 120 between in and the structure supporting it.

In various exemplary embodiments, the toilet continuously measures weight load on both the seat 200 and the foot platform 120. In a preferred embodiment, the system weighs the user on the foot platform 120 prior to the user placing any weight against the seat 200 and records that as the user's total weight. In some embodiments, the system calculates a user total weight using both the seat weight sensors 203 and foot platform weight sensors 121 after the user seated with the seat 200 fully lowered. In various exemplary embodiments, the system calculates the percentage of user body weight on the foot platform 120. The angle of the seat 200 is limited from raising based on the percentage of weight on the foot platform 120. This prevents the seat 200 from tipping to an angle that will not support the user's weight prior to the user being on their feet.

Referring still to FIG. 8, the foot platform 120 is adapted to provide for imaging sensors 134 to examine a user's feet. Imaging sensors 134 are positioned above, to the sides, and below the user's feet on the arms 130, crossbar 132, and base 133. The imaging sensors 134 may be visible light, infrared, near infrared, thermal, and/or 3D sensors. A foot platform and imaging sensors of this type are described in U.S. patent application Ser. No. 16/578,118 filed on 20 Sep. 2019, which is incorporated herein by reference in its entirety.

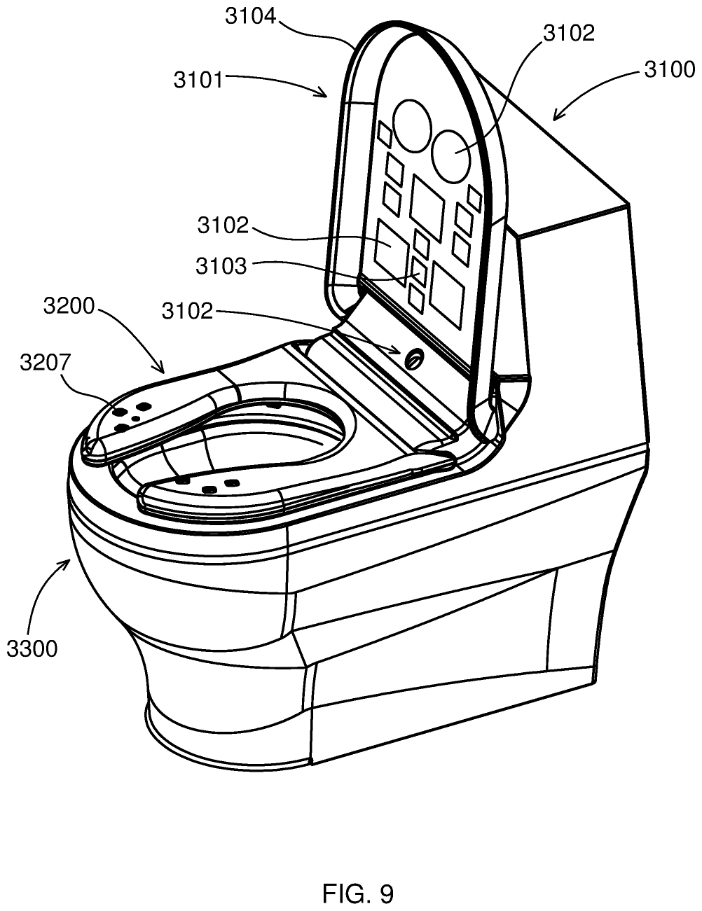

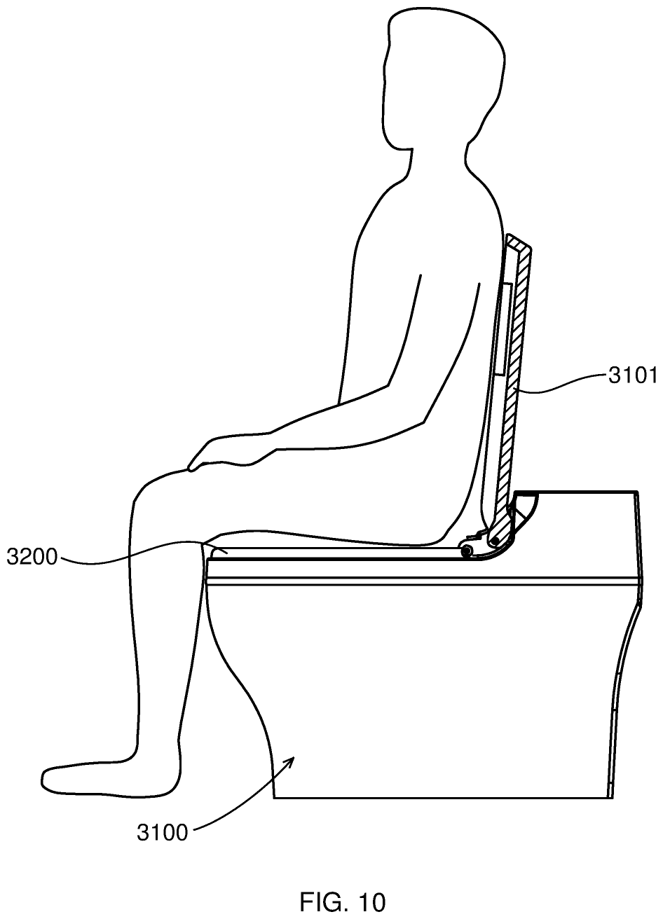

Referring to FIG. 9, an exemplary embodiment of an analytical toilet 3100 is shown. The seat 3200 includes one or more health and wellness sensors 3207. The lid 3101 includes one or more sensors 3102. The sensors 3102 may interact with the user's back while using the toilet, as shown in FIG. 10, or may be collecting data with the lid 3101 closed. The interior of the bowl 3300 may be illuminated with visual, infrared, near infrared, ultraviolet, or other wavelengths by light sources 3103.

In various exemplary embodiments, the lid 3101 includes a seal 3104 that forms an airtight seal creating an airtight chamber including the bowl 3300. In such embodiments, the toilet may include sensors for testing the air in the chamber and/or passages for drawing air out of the chamber for testing elsewhere in the system. Such sensors or tests may include electrocardiography, CCD, MOS/CMOS, spectrometers, chromatographs, FET, nano-FET, MOSFET, mass spectrometers, electrodes, microphones, load cells, pressure gauges, PPG, thermometers (including IR and thermocouples), rheometers, durometers, pH detectors, and scent detectors. Drawing air out of the chamber may also aid in drying the bowl between uses to reduce the risk of cross-sample contamination. The air passages may also be used to increase pressure in the airtight chamber. This may be advantageous in preparing feces for sampling or other purposes. In other embodiments, the seal may be water-tight or splash-proof.

In various exemplary embodiments, the inner surface of the lid 3101 may include an ultraviolet light source to disinfect the seat and/or bowl.

All patents, published patent applications, and other publications referred to herein are incorporated herein by reference. The invention has been described with reference to various specific and preferred embodiments and techniques. Nevertheless, it is understood that many variations and modifications may be made while remaining within the spirit and scope of the invention.

What is claimed is:

1. An analytical toilet comprising:
a bowl for receiving excreta;
a health and wellness sensor; and
a processor receiving and analyzing data from the health and wellness sensor;
wherein the processor analyzes data from the health and wellness sensor to detect when a user is positioned to use the toilet.

2. The analytical toilet of claim 1 further comprising a data connection through which the processor may transmit and receive data.

3. The analytical toilet of claim 2 wherein the processor analyzes data from the health and wellness sensor to identity a user.

4. The analytical toilet of claim 3 wherein the health and wellness sensor is an imaging sensor.

5. The analytical toilet of claim 3 wherein the identity of the user is determined by comparing data from the health and wellness sensor to a database of known users.

6. The analytical toilet of claim 1 wherein the health and wellness sensor is incorporated into a peripheral device.

7. The analytical toilet of claim 6 wherein the peripheral device is a scale.

8. The analytical toilet of claim 1 wherein the health and wellness sensor comprises one or more of electrocardiography, CCD, MOS/CMOS, spectrometers, chromatographs, FET, nano-FET, MOSFET, mass spectrometers, electrodes, microphones, load cells, pressure gauges, PPG, thermometers, rheometers, durometers, pH detectors, and scent detectors.

9. The analytical toilet of claim 1 wherein the health and wellness sensor is located in one or more of the bowl, seat, lid, foot platform, handles, and frame.

10. The analytical toilet of claim 1 wherein the health and wellness sensor is an imaging sensor.

11. The analytical toilet of claim 10 wherein the imaging sensor is one or more of CCD, MOS/CMOS, visible light camera, IR camera near IR camera, 3D camera, or UV camera.

12. The analytical toilet of claim 10 wherein the imaging sensor use one or more of light or sound waves.

13. The analytical toilet of claim 12 wherein the imaging sensor uses ultrasound.

14. The analytical toilet of claim 12 wherein the imaging sensor uses infrared, near-infrared, visible, or ultraviolet light.

15. The analytical toilet of claim 1 further comprising a detector capable of detecting an identifying item of the user.

16. The analytical toilet of claim 15 wherein the identifying item is a phone, identification, credit or debit card, RFID tag, or other personal electronic device.

17. The analytical toilet of claim 1 wherein the processor detects a change in a health and wellness sensor output indicative of a user and activates additional health and wellness sensors based on the position of the user.

18. The analytical toilet of claim 17 wherein the toilet system activates health and wellness sensors based on the identity of the user.

19. The analytical toilet of claim 17 wherein the health and wellness sensor is located in one or more of the bowl, seat, lid, foot platform, handles, and frame.

20. The analytical toilet of claim 1 wherein the data for a user is tracked over time for changes.
